(19) United States
(12) Patent Application Publication
(10) Pub. No.: US 2016/0213434 A1
(43) Pub. Date: Jul. 28, 2016

(54) Robot Comprising A Tool

(71) Applicant: KUKA LABORATORIES GMBH, Augusburg (DE)

(72) Inventors: Sebastian Lohmeier, Munchen (DE); Wolfgang Schober, Pottmes (DE)

(73) Assignee: KUKA ROBOTER GMBH, Augsburg (DE)

(21) Appl. No.: 14/787,955

(22) PCT Filed: Mar. 11, 2014
(86) PCT No.: PCT/EP2014/000646

§ 371 Date: Feb. 12, 2016

(30) Foreign Application Priority Data
May 2, 2013 (DE) 10 2013 007 597.1

(51) Int. Cl. A61B 34/30; A61B 17/00

(52) U.S. Cl. CPC A61B 34/30; A61B 17/00234; A61B 2017/00336; A61B 2017/00991

(57) ABSTRACT

A robot according to the invention comprises a tool, in particular a surgical instrument, said tool comprising a shaft having a distal joint assembly with at least one degree of freedom. The robot also comprises a protective cover that can be displaced from a distal position into a proximal position on the shaft. In the distal position, said protective cover accommodates the joint assembly, and when the protective cover is in the proximal position the joint assembly projects distally out of the protective cover.

### TECHNICAL FIELD

The present invention concerns a robot with a tool, in particular a surgical instrument, comprising a shaft with a distal joint assembly with at least one degree of freedom, which can be accommodated in a protective cap, a corresponding protective cap and a method for handling a tool of such a robot.

### BACKGROUND

In robotic surgery, which is minimally invasive, one or more robots respectively guide a surgical instrument, the shaft of which partly reaches into the interior of a patient through a natural or man-made access, in particular a trocar sleeve. So as to not have there only three rotational degrees of freedom around the access point and a translational degree of freedom in the direction of insertion, the shaft exhibits a distal joint assembly with one or more proximally actuated degrees of freedom, via which an end effector in the interior of the patient can additionally be actuated, for example pliers can be opened and closed, and/or adjusted, for example, pivoted. Before and/or after the intracorporeal use of the surgical instrument, in particular in hectic OR situations, the end effector and the distal joint assembly of the robot-guided instrument can come into contact with the surroundings.

The task of the present invention is to improve the operation of a robot guiding a tool, in particular a surgical instrument.

### SUMMARY

In one design, a robot according to one aspect of the present invention comprises kinematics with at least six, preferably at least seven, degrees of freedom, in particular rotational degrees of freedom, between a robot base and a tool flange, to which, preferably releasably, a robot-guided tool, in particular a surgical instrument, is attached. The tool or instrument comprises a preferably hollow shaft, which in one design is, at least substantially, configured to be tubular, preferably with an at least partially circular and/or at least partially straight, in particular polygonal, outer contour. Additionally or alternatively, the shaft can be divided into jointed sections and/or comprises one or more flexible sections.

At a distal end of the shaft, the shaft comprises a distal joint assembly with one or more, preferably proximally actuated, degrees of freedom, in particular at least one rotational degree of freedom for pivoting against a shaft (longitudinal) axis, at least one translational degree of freedom and/or at least one functional degree of freedom for actuating, in particular opening, closing and/or adjusting, an end effector of the tool shaft. Additionally or alternatively, the distal end of the shaft can also comprise one or more flexible sections, which in one further development are actuatable, in particular proximally, in particular able to be angled to represent degrees of freedom, in particular to move an end effector. In the sense of the present invention, such elastic or distributed joints are also referred to as joints of a distal joint assembly. Accordingly, degrees of freedom of the distal joint assembly, in particular rigid body degrees of freedom and/or elastic degrees of freedom, can be angling of a flexible section or segment, for example. The end effector can be configured in one-piece, in particular as a scalpel, needle or the like, or in multiple parts, in particular as scissors, clamps, and pliers. It can have an opening for taking in and/or releasing fluids, in particular gases and/or liquids, and/or electromagnetic radiation, in particular an aspirator, an injection nozzle, or the optics of a camera or a laser. Distal in the sense of the present invention is in particular an end of the shaft further away from the robot, a direction from the robot towards this end, particularly towards or beyond the end effector, or a position more distant from the robot, in particular relative to a position that is proximal or closer to the robot. Accordingly, proximal is in particular an end of the shaft closer to the robot, a direction from the end effector towards or beyond the robot, or a position closer to the robot, in particular relative to a position that is distal or further away from the robot. In one design, the shaft is provided or equipped for partial insertion through a natural or man-made access, in particular a trocar sleeve, into the interior of a patient, and/or comprises a proximal drive for actuating at least one, preferably all, degrees of freedom of the distal joint assembly. In addition to the degree or degrees of freedom of the distal joint assembly, the shaft can comprise other, in particular distal or proximal, degrees of freedom. Accordingly, a distal joint assembly is in particular understood to be an arrangement of one or more distal joints between a shaft pipe and an end effector connected to it in an articulated manner.

According to one aspect of the present invention, a protective cap is movably disposed on the shaft, in particular guided, and can be moved from a distal to a proximal position on the shaft, whereby in the distal position the protective cap accommodates the joint assembly, preferably at least substantially encircling the outer contour casing surface in a sleeve or cage-like manner, and whereby the joint assembly protrudes distally, in particular completely, from the protective cap when the protective cap is in the proximal position. In one design, the protective cap in the distal position additionally also encircles, at least substantially, the end effector. Correspondingly, according to one aspect, in particular the insertion of an end effector into a patient, the protective cap is moved from the distal to the proximal position on the shaft of the tool prior to being used. In a further development, this can be carried out by the robot itself, in particular by combining or connecting the protective cap with a sleeve, in particular a trocar sleeve. The robot can, in particular, position the distal cap of the tool at the trocar sleeve and move them proximally along the shaft by inserting the tool shaft. After use of the tool, the protective cap in one design is moved, preferably automatically, along the shaft of the tool from the proximal back to the distal position. This protective cap in one design can in particular be prestressed in the distal direction.

In one design, the robot comprises a sleeve, in particular a trocar sleeve, which can be combined or connected to the protective cap, preferably in a releasable manner. In one design, the sleeve and protective cap can be combined or connected in a form-fitting manner. The protective cap and the sleeve can in particular comprise a, preferably centering, guide. In one design, the protective cap comprises a preferably conical outer contour on a distal face and the sleeve comprises a hereto at least substantially complementary inner contour on a proximal face. Similarly, the sleeve can comprise a, preferably conical, outer contour on a proximal face and the protective cap can comprise a hereto at least substantially complementary inner contour on a distal face. Additionally or alternatively, the guide can be symmetrical or asymmetrical to the longitudinal axis of the shaft, so as to allow or preclude a connection of protective cap and sleeve in orientations that are rotated relative to one another. In one design, sleeve and protective cap are connected in a torque-proof manner, in another in a rotatable manner.

Additionally or alternatively, sleeve and protective cap can be combined or connected in a force-locking manner. In one design, a magnet assembly with one or more permanent and/or electromagnets is disposed on the protective cap, in particular at a distal end of the protective cap, and/or on the sleeve, in particular at a proximal end of the sleeve, so as to magnetically connect sleeve and protective cap to one another, in particular in a switchable manner. Additionally or alternatively, sleeve and protective cap can be combined or connected in a frictionally engaged manner.

In one design, the protective cap comprises a distal cover, which covers the end effector in distal direction when the cover is closed. The distal cover can, in particular, be configured to be reversible, openable and reclosable. For this purpose, the distal cover can comprise one or more pivotable and/or movable flaps, which in a further development are mounted, pivotably or parallel and/or transverse to the shaft axis to the outside or away from the shaft axis and/or inwards or toward the shaft axis, in a movable manner. Additionally or alternatively, the distal cover can comprise one or more segments, which in a further development are elastically deformable outward and/or inward. At thin places in the material or at film hinges, the segments can pivot as a whole or as flaps. Additionally or alternatively, the segments can themselves be elastically deformed. The segments are preferably separated from one another by thin places in the material or by continuous slits. Accordingly, the distal cover can be opened in particular by moving the protective cap in proximal direction through the end effector. The cover can be reversibly openable and reclosable; in particular segments or flaps, which fold away elastically, close automatically when the protective cap is brought back into the distal position. Additionally or alternatively, the end effector can also open the cover in sections, particularly by partitioning the cover into segments along thin places in the material, whereby, in a further development, these segments can reset elastically as well. In one design, the cover is or can be attached to the protective cap in a releasable, in particular releasable and refastenable, manner.

In one design, the protective cap can be locked in the distal position, in particular to avoid unintentional exposure of the distal joint assembly. Additionally or alternatively, the protective cap can be locked in the proximal position in the same or in a different manner, in particular to avoid unintentionally taking in the distal joint assembly and/or strain on a sleeve, in particular a trocar sleeve, by the distal prestressed protective cap and/or to improve the cleaning of the instrument. Additionally or alternatively, the protective cap can be locked in one or more discrete intermediate positions or also continuously or anywhere between the distal and the proximal position in the same or in a different manner.

In a further development, the protective cap is or can be locked in the distal position, the proximal position, and/or at least one intermediate position, in a releasable manner. This can be done in particular in a form-fitting manner, in particular by twisting the protective cap on the shaft, somewhat in the manner of a bayonet catch, or by a snap lock, in which one or more hooks on the protective cap or the shaft engage behind corresponding indentations on the shaft or the protective cap. Additionally or alternatively, the protective cap is or can be locked in a force-locking, in particular frictionally engaged and/or magnetic, manner.

In one design, the protective cap in the distal position, in the proximal position and/or between the distal and the proximal position is guided on the shaft in a movably torque-proof manner. For this purpose, the shaft and/or the protective cap can comprise one or more radial projections, which are guided in corresponding longitudinal grooves in the protective cap or the shaft. Additionally or alternatively, the shaft can comprise a not fully circular or not completely round, in particular at least partially even, preferably polygonal outer contour and the protective cap can comprise a thereto complementary bore. In general, in one design, the protective cap can comprise, particularly proximally, a bore through which the shaft passes to guide the protective cap movably on the shaft.

In one design, the protective cap comprises a convergent, in particular distal, insertion opening. This allows the distal joint kinematics to, at least substantially, automatically be aligned to the shaft axis during the return movement of the protective cap into the distal position. In a meridional section through the shaft axis, the convergent insertion opening can in particular comprise a bevel or a convex curvature, or the convergent insertion opening can be configured as a chamfer or rounding, in particular elliptical or as a radius.

In one design, the protective cap is captively bound to the shaft and/or the robot. For this purpose, in one design, a bore of the protective cap passed through by the shaft can be closed or uncut across its periphery and the shaft can comprise at least one radial projection, in particular a radial collar, the outer diameter of which is larger than the inner diameter of the bore and which thus acts as a stop, preventing, or at least impeding, the distal removal the protective cap. Additionally or alternatively, the protective cap can also be bound to the shaft and/or the robot by a, preferably prestressed, traction mechanism, in particular a safety cable.

In one design, the protective cap is, at least partially, made of an elastic material. It can be manufactured entirely or in part of metal and/or one or more plastics, in particular thermoplastics, silicone and/or, preferably thermoplastic, elastomers.

In one design, the protective cap is configured in one piece or formed integrally. The protective cap can also be configured in multiple parts. It can in particular comprise a guide sleeve to guide it on the shaft and a cover sleeve to accommodate the distal joint assembly. In one design, the guide and the cover sleeves can be combined or connected to one another in a releasable manner, in particular in a form-fitting and/or force-locking, in particular frictionally engaged and/or magnetic, manner.

In one design, the entire protective cap or one or more parts of the protective cap, in particular a guide sleeve and/or a cover sleeve, can be removed from the shaft. In particular for this purpose, in one design, the protective cap comprises in particular the guide sleeve and/or the cover sleeve, in one design an in particular continuous slit, which in one design extends from a proximal to a distal face. In another design, one or more slits extend from a bore of the protective cap passed through by the shaft, in particular the guide sleeve, so that the protective cap or guide sleeve can be removed from the shaft through elastic widening of the bore.

In one design, the protective cap can, at least substantially, completely cover the joint assembly in the distal position in peripheral direction, or comprise an, at least substantially, closed casing surface. Additionally or alternatively, the protective cap can, at least substantially, completely cover the joint assembly in the distal position in distal direction, or comprise an, at least substantially, closed distal cover. Additionally or alternatively, the protective cap can, at least substantially, in particular up to a bore of the protective cap passed through by the shaft, completely cover the joint assembly in the distal position in proximal direction, or comprise an, at least substantially, closed proximal face. The protective cap can also be configured in a cage-like manner, and comprise a casing surface, distal and/or proximal face with one or more openings respectively.

### BRIEF DESCRIPTION OF THE DRAWINGS

Additional advantages and features result from the dependent claims and the design examples. For this purpose, the figures show, in part schematically:

FIG. 1: a section of a shaft of a surgical instrument with an end effector and a protective cap in a distal position and, at a distance to it, a trocar sleeve (FIG. 1(a)—and with the protective cap in a proximal position and the trocar sleeve (FIG. 1(b) connected to it) of a robot according to one design of the present invention;

FIG. 2: one-piece protective caps according to two designs of the present invention;

FIG. 3: a guide sleeve and a cover sleeve of a protective cap according to one design of the present invention in a plan view (FIG. 3(a)) and sectional view (FIG. 3(b));

FIG. 4: protective caps according to two designs of the present invention with different convergent insertion openings;

FIG. 5: a protective cap and a trocar sleeve that can be connected to it according to two designs of the present invention;

FIG. 6: protective caps according to four designs of the present invention with different covers;

FIG. 7: a protective cap according to one design of the present invention that is rotatably guided and captively bound to the shaft;

FIG. 8: a protective cap according to one design of the present invention that is rotatably guided and captively bound to the shaft;

FIG. 9: a protective cap according to one design of the present invention that is guided in a torque-proof manner and captively bound to the shaft;

FIG. 10: a protective cap according to one design of the present invention that is guided in a torque-proof manner and captively bound to the shaft;

FIG. 11: a protective cap according to one design of the present invention that is captively bound to the shaft;

FIG. 12: a protective cap according to one design of the present invention locked on the shaft in the distal position;

FIG. 13: a protective cap according to one design of the present invention locked on the shaft in the distal position;

FIG. 14: a protective cap according to one design of the present invention locked on the shaft in the distal position;

FIG. 15: a shaft of a surgical instrument with an end effector, a protective cap, as well as a trocar sleeve connected to it, of a robot according to one design of the present invention;

FIG. 16: a shaft of a surgical instrument with an end effector, a distal prestressed protective cap and a trocar sleeve of a robot according to one design of the present invention; and

FIG. 17: a shaft of a surgical instrument with an end effector, a distal prestressed protective cap and a trocar sleeve of a robot according to one design of the present invention.

### DETAILED DESCRIPTION

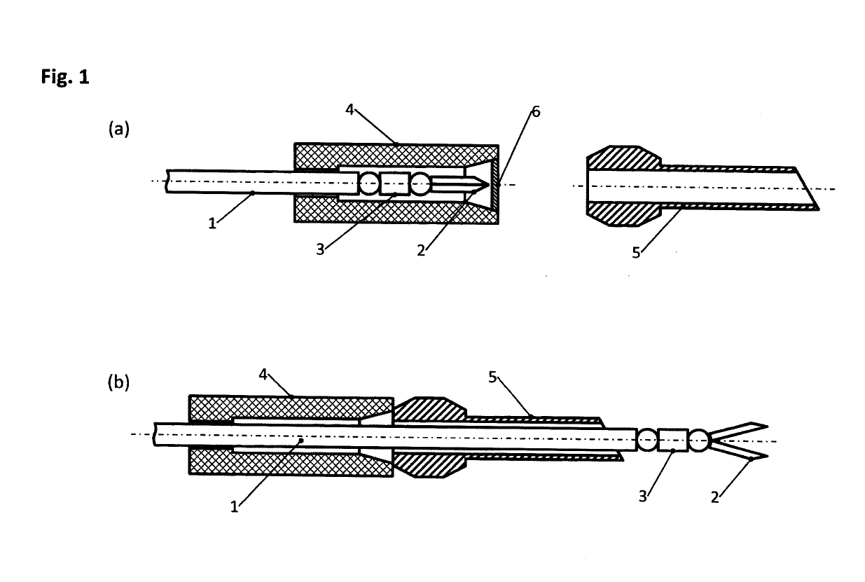

FIG. 1 shows a section of a shaft 1 of a surgical instrument of a robot (left—not shown in FIG. 1) according to one design of the present invention with an end effector 2 and a distal joint assembly 3 with two rotational degrees of freedom, as well as a protective cap 4, which can be moved from the distal position on the shaft shown in FIG. 1(a) to a proximal position shown in FIG. 1(b), by the robot placing the protective cap onto a trocar sleeve 5 and guiding the instrument shaft into or through them (from left to right in the sequence of figures FIG. 1(a)-FIG. 1(b)).

As can be seen in the section of FIG. 1, in the distal position (FIG. 1(a)), the protective cap accommodates the distal joint assembly and the end effector; in the proximal position (FIG. 1(b)), the joint assembly and the end effector protrude distally from the protective cap and the trocar sleeve (to the right in FIG. 1).

Prior to use of the surgical instrument in a patient, the robot moves the protective cap from the distal into the proximal position on the shaft of the instrument through contact of the trocar sleeve, i.e. connecting to the trocar sleeve, by pushing the instrument shaft distally into the trocar sleeve or further through this trocar sleeve. After use, the robot moves the protective cap connected to the trocar from the proximal to the distal position sleeve on the shaft of the tool, by pulling the instrument shaft proximally out of the trocar sleeve or away from the trocar sleeve.

In this way, the surroundings or the extracorporeal distal joint assembly 3 and the end effector 2 are protected by the protective cap 4.

The protective cap can exhibit a distal cover 6, which in one design is or can be removed in sections prior to the first connection with the trocar sleeve. In another design, the distal cover can also be designed as a pivotable flap, a membrane to be pierced or the like.

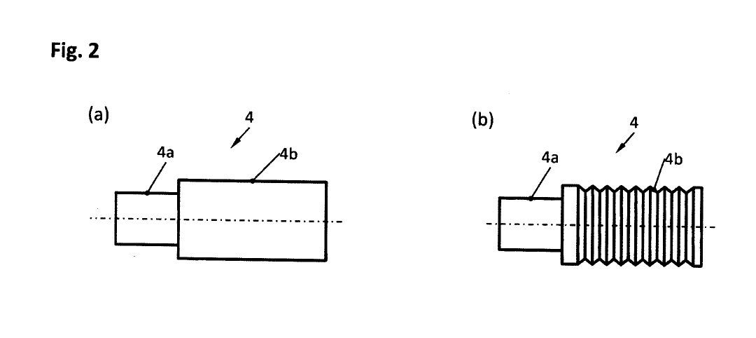

In a representation corresponding to FIG. 1, FIG. 2 shows one-piece protective caps according to two designs of the present invention. Corresponding elements are identified with the same reference signs, so that only the differences to the other depicted designs are discussed in the following, while we otherwise refer to their descriptions.

In the design of FIG. 2(a), the one-piece, tubular protective cap 4 exhibits a proximal (on the left in FIG. 2) section 4a with a smaller diameter for guiding the protective cap on the shaft (not depicted), and a distal (on the right in FIG. 2) section 4b with a larger diameter to represent a larger insertion opening and/or to accommodate the distal joint assembly and the end effector (not depicted). As can be seen in the section of FIG. 1, there is a bore in the proximal section 4a, through which the shaft passes. The protective cap is movably guided on the shaft in this manner. The protective cap can be removable from the shaft and, for this purpose, exhibit in particular a slit extending from this bore (not depicted), which allows a radial widening and distal withdrawal of the protective cap from the shaft. The protective cap can also be captively bound to the shaft. The protective cap can in particular be manufactured of plastic, preferably a hard or soft thermoplastic, thermoplastic elastomer, silicone and/or metal.

In the design of FIG. 2(b), the distal section 4b is configured as corrugated bellows.

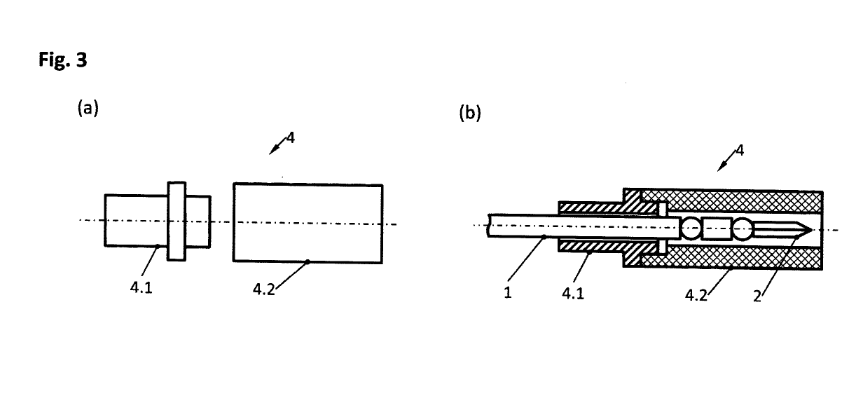

In a representation corresponding to FIG. 1, 2, FIG. 3 shows a guide sleeve 4.1, and a covering sleeve 4.2 of a cap according to one design of the present invention that can be connected to it (FIG. 3(a)), or is connected to it (FIG. 3(b)), in a plan view (FIG. 3(a)) and in a sectional view (FIG. 3(b)). Corresponding elements are identified with the same reference signs, so that only the differences to the other depicted designs are discussed in the following, while we otherwise refer to their descriptions.

The guide sleeve 4.1 is removable or guided on the shaft in a captive and movable manner; the cover sleeve 4.2 is connected to the guide sleeve in a form-fitting and/or frictionally engaged manner, for example by an interference fit, so that they can be withdrawn from the guide sleeve and the instrument shaft.

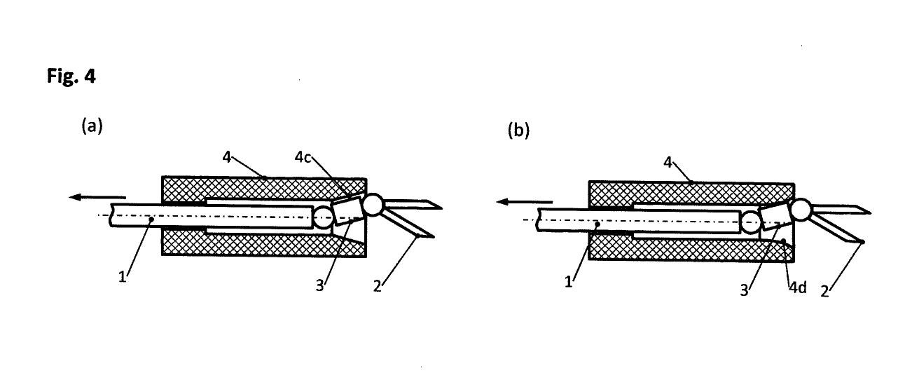

In a representation corresponding to FIG. 1, 3(b), FIG. 4 shows protective caps 4 according to two designs of the present invention. Corresponding elements are identified with the same reference signs, so that only the differences to the other depicted designs are discussed in the following, while we otherwise refer to their descriptions.

In FIG. 4 the protective caps 4 are in an intermediate position between the distal position, in which they accommodate the end effector and the distal joint assembly (see. FIG. 1(a), 3(b)), and the proximal position (see FIG. 1(b)), in which the distal joint assembly protrudes entirely from the protective cap.

Each of the protective caps 4 distally exhibits (right in FIG. 4) a convergent insertion opening 4c and 4d, a chamfer 4c in the design of FIG. 4(a), an elliptic rounding 4d in the design of FIG. 4(b). In this way, as indicated in FIG. 4 by a motion arrow, when the protective cap 4 is moved into the distal position, the distal joint assembly is, at least substantially, aligned with the shaft axis, and the joint assembly is thereby inserted through the convergent insertion opening into the protective cap and accommodated therein.

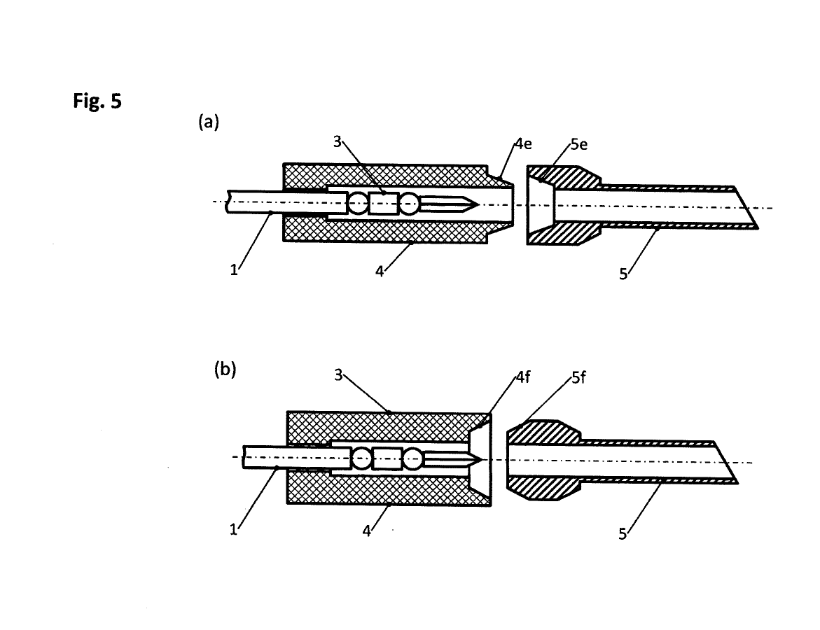

In a representation corresponding to FIG. 1(a), FIG. 5 shows protective caps 4 and therewith connectable trocar sleeves 5 according to two designs of the present invention. Corresponding elements are identified with the same reference signs, so that only the differences to the other depicted designs are discussed in the following, while we otherwise refer to their descriptions

The protective caps 4 and trocar sleeves 5 exhibit a centering guide. For this purpose, in the design of FIG. 5(a), the protective cap 4 exhibits a conical outer contour, in particular an outer cone 4e, on a distal face (on the right in FIG. 5), and the sleeve 5 exhibits a thereto complementary inner contour, in particular an inner cone 5e, on a proximal face (on the left in FIG. 5). In the design of FIG. 5(b), the sleeve 5 conversely comprises a conical outer contour in the form of an outer cone 5f on a proximal face, and the protective cap 4 comprises a thereto complementary inner contour in the form of an inner cone 4f on a distal face.

The protective cap 4 and the trocar sleeve 5 can be or are connected to one another in a torque-proof, in particular a frictionally engaged, manner. They can be or are connected to one another in a rotatable manner as well, in particular to allow rotation of the instrument shaft relative to the trocar sleeve, even if the protective cap is connected to the instrument shaft in a torque-proof manner.

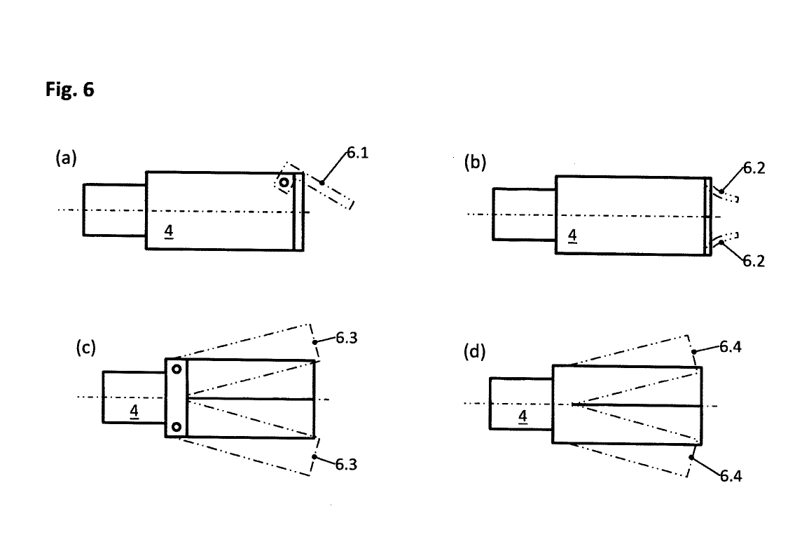

In a representation corresponding to FIG. 2, 3(a), FIG. 6 shows protective caps 4 according to four designs of the present invention with different covers. Corresponding elements are identified with the same reference signs, so that only the differences to the other depicted designs are discussed in the following, while we otherwise refer to their descriptions.

The distal cover of the design of FIG. 6(a) is configured as a one-piece flap 6.1, which can be pivoted outward or away from the shaft axis indicated with the dot-dashed line. The distal cover of the design of FIG. 6(b) comprises multiple segments 6.2, which are elastically deformable into the configuration indicated by the dot-dashed line or can be pivoted outward. To do this, thin places in the material between the segments 6.2 can be separated when it is first moved. The distal cover of the design of FIG. 6(c) comprises two flaps 6.3 that can be pivoted to the outside into the configuration indicated by the dot-dashed line in an articulated manner. The distal cover of the design of FIG. 6(d) comprises two segments 6.4, which are elastically deformable to the outside into the configuration indicated by the dot-dashed line. To do this, thin places in the material between the segments 6.4 can be separated when it is first moved.

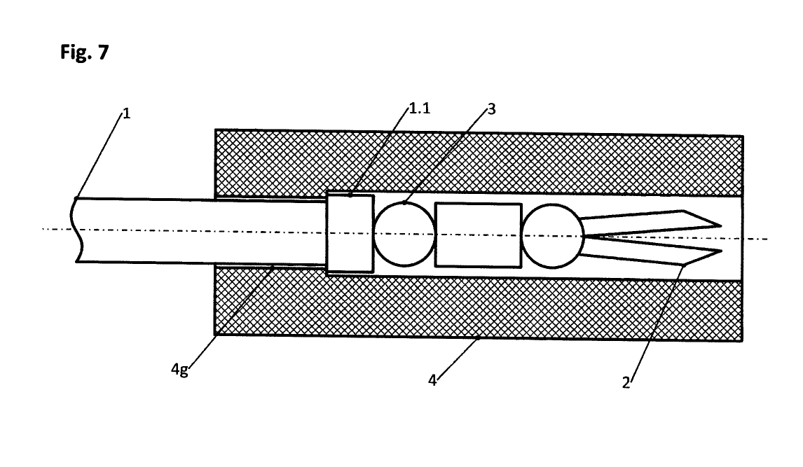

In a representation corresponding to FIG. 1(a), 3(b), 5, FIG. 7 shows a protective cap that is rotatably guided and captively bound on the shaft according to one design of the present invention. Corresponding elements are identified with the same reference signs, so that only the differences to the other depicted designs are discussed in the following, while we otherwise refer to their descriptions.

For this purpose, shaft 1 comprises a radial collar 1.1 proximal to the distal joint assembly 3, the outer diameter of which is greater than the inner diameter of the circular bore 4g of the protective cap 4, through which the cylindrical shaft 1 passes with play.

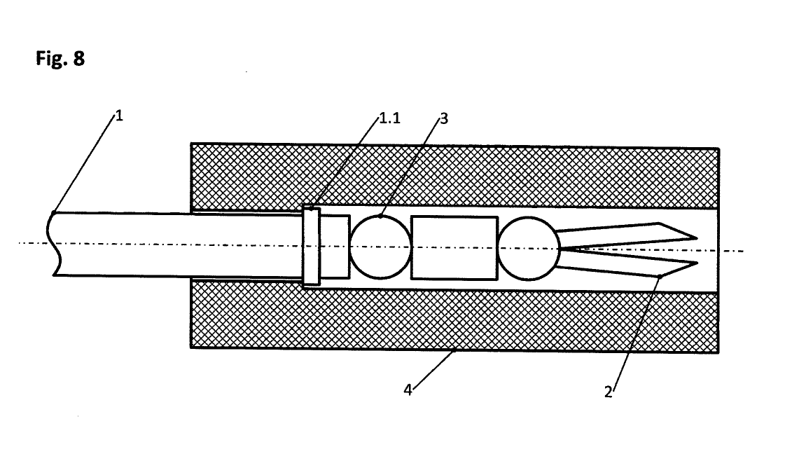

In a representation corresponding to FIG. 7, FIG. 8 shows a protective cap that is rotatably guided and captively bound to the shaft according to one design of the present invention. Corresponding elements are identified with the same reference signs, so that only the differences to the other depicted designs are discussed in the following, while we otherwise refer to their descriptions.

In the design of FIG. 8, the radial collar 1.1 is proximally spaced from the distal joint assembly 3.

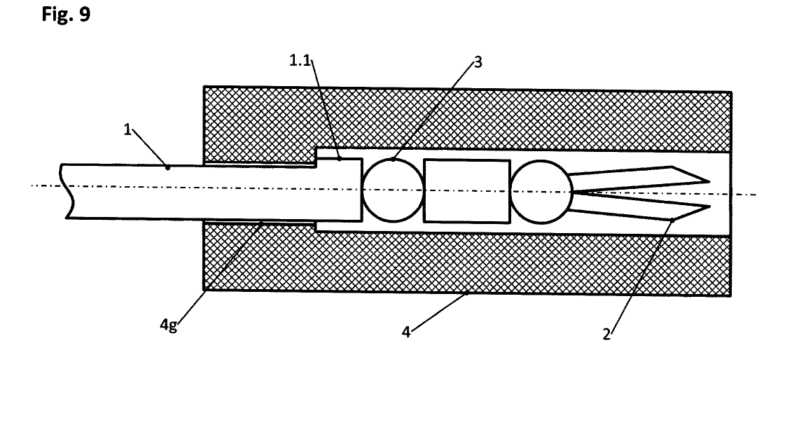

In a representation corresponding to FIG. 7, 8, FIG. 9 shows a protective cap that is captively bound and guided on the shaft in a torque-proof manner according to one design of the present invention. Corresponding elements are identified with the same reference signs, so that only the differences to the other depicted designs are discussed in the following, while we otherwise refer to their descriptions.

To guide the protective cap on the shaft in a torque-proof manner, the bore 4g and the shaft 1 of the design of FIG. 9 comprise, at least in part (above in FIG. 9), an even contour; here in the form of a flattening of the otherwise circular contour. In a variation, the bore and the shaft can also comprise complementary polygonal, in particular triangular, rectangular or more-sided, inner and outer contours. Other not fully circular, or not completely round, contours, e.g. elliptical contours, a polygonal shaft profile or the like, are possible as well.

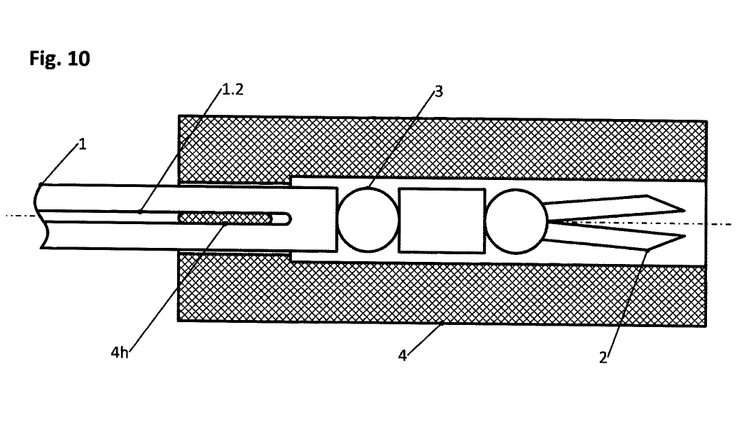

In a representation corresponding to FIG. 9, FIG. 10 shows a protective cap that is captively bound and guided on the shaft in a torque-proof manner according to one design of the present invention. Corresponding elements are identified with the same reference signs, so that only the differences to the other depicted designs are discussed in the following, while we otherwise refer to their descriptions.

For this purpose, a longitudinal groove 1.2, in particular in the form of a corrugation, is configured in the shaft 1, in which a radial projection 4h of the protective cap is guided in a movable manner. The radial projection 4h can be configured as an integral part of the rest of the protective cap, or as a separate slide that is connected to the rest of the protective cap. By means of the projection 4h, guided in the groove 1.2, which is closed at the end or distally, the protective cap 4 is guided on the shaft 1 both captively bound as well as in a torque-proof manner.

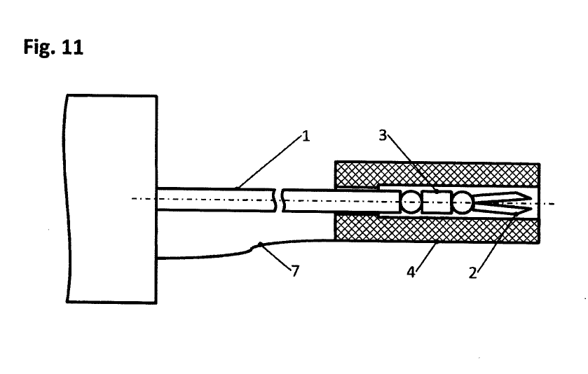

FIG. 11 shows a part of a surgical instrument of a robot (on the left, not shown in FIG. 1) with a shaft 1, according to one design of the present invention, with an end effector 2 and a distal joint assembly 3 with two rotational degrees of freedom, as well as a protective cap 4. Corresponding elements are identified with the same reference signs, so that only the differences to the other depicted designs are discussed in the following, while we otherwise refer to their descriptions.

In the design of FIG. 11, the protective cap 4 is captively bound on the shaft by means of a safety cable 7.

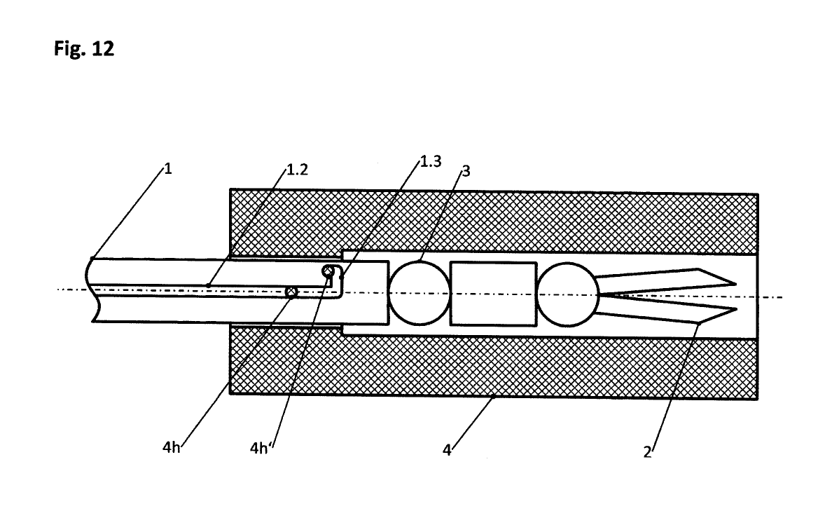

In a representation corresponding to FIG. 7-10, FIG. 12 shows a protective cap that is captively bound on the shaft according to one design of the present invention. Corresponding elements are identified with the same reference signs, so that only the differences to the other depicted designs are discussed in the following, while we otherwise refer to their descriptions.

In the design of FIG. 12, the protective cap can be locked in the distal position in a releasable, form-fitting manner. For this purpose, the groove 1.2 exhibits a leg 1.3 in the shaft 1 in peripheral direction, into which the radial projection 4h can be inserted by twisting the protective cap 4 on the shaft 1 and via which the protective cap 4 can be locked in the manner of a bayonet catch. For clarification, the projection 4h is drawn into both a shaft axis parallel section of the groove 1.2, and into the leg 1.3, where it is identified as 4h′. In a not depicted variation, instead of the bayonet catch, a form-fitting locking mechanism, in particular a spring-loaded catch, or a frictionally engaged clamp, can be provided to lock the protective cap.

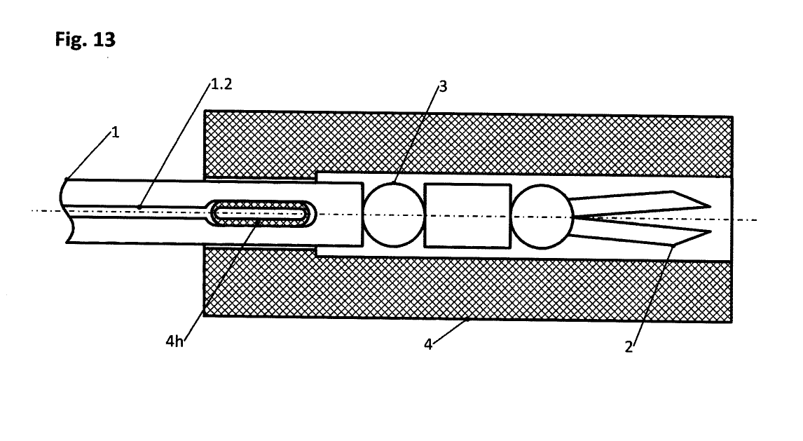

FIG. 13 shows a further development of the design illustrated with reference to FIG. 10. Corresponding elements are identified with the same reference signs, so that only the differences to the other depicted designs are discussed in the following, while we otherwise refer to their descriptions.

In the design of FIG. 13, the movable protective cap 4 is locked on the shaft by means of a cross-sectional widening of the groove 1.2. For this purpose, the projection 4h is configured to be elastic, so that it expands in the cross-sectional widening and thus provides resistance to movement out of the cross-sectional widening.

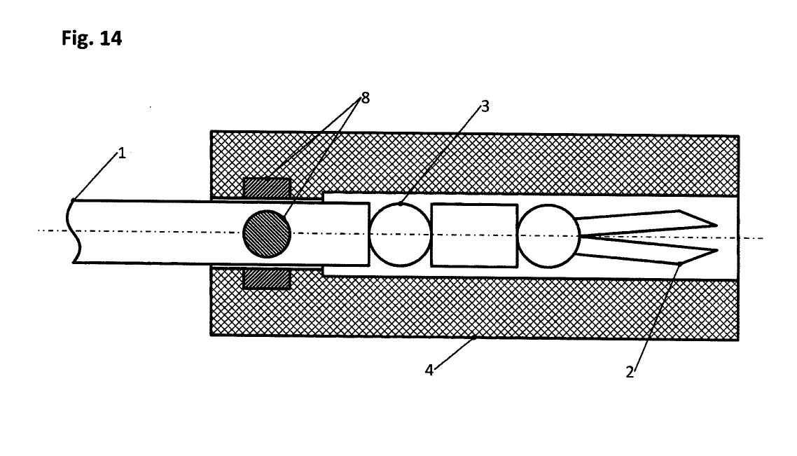

In a representation corresponding to FIG. 7-10, 12-13, FIG. 14 shows a protective cap that is locked on the shaft according to one design of the present invention. Corresponding elements are identified with the same reference signs, so that only the differences to the other depicted designs are discussed in the following, while we otherwise refer to their descriptions.

In the design of FIG. 14, the shaft 1 and/or the protective cap 4 comprise magnets 8, which lock the protective cap in a predetermined position on the shaft. As in the design of FIG. 14, this can be its distal position. Additionally or alternatively, in an analogous manner, the protective cap can be magnetically locked on the shaft in a different predetermined position, in particular in the proximal position. The locking mechanisms, illustrated above with reference to FIG. 12, 13, can also be provided additionally or alternatively to a locking mechanism in the distal position, for the purpose of locking in at least one other, in particular the proximal, position.

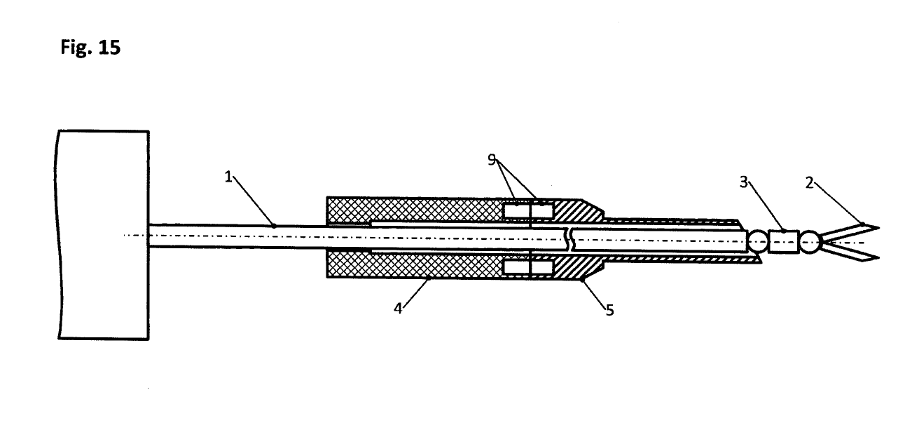

In a representation corresponding to FIG. 11, FIG. 15 shows a section of a surgical instrument of a robot according to one design of the present invention. Corresponding elements are identified with the same reference signs, so that only the differences to the other depicted designs are discussed in the following, while we otherwise refer to their descriptions.

In the shown proximal position, the protective cap 4 is connected to the trocar sleeve 5 temporarily and in a repeatedly releasable manner. A locking mechanism 9, which can in particular be configured as a form-fitting snap or catch mechanism, a latching tapered connection, or to be magnetic, is schematically indicated for this purpose in FIG. 15

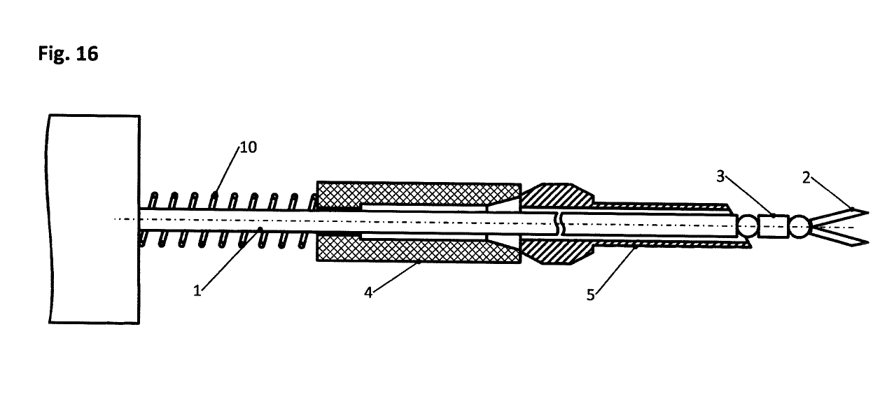

In a representation corresponding to FIG. 11, 15, FIG. 16 shows a section of a surgical instrument of a robot according to one design of the present invention. Corresponding elements are identified with the same reference signs, so that only the differences to the other depicted designs are discussed in the following, while we otherwise refer to their descriptions.

In the design of FIG. 16, the protective cap is prestressed in distal direction (to the right in FIG. 16) by a spring 10. When the robot pulls the instrument shaft proximally from the trocar sleeve or away from it, the spring 10 automatically pushes the protective cap 4 back into the distal position. In addition, during operation, the compressed spring 10 presses the proximally positioned cap against the trocar sleeve.

In a representation corresponding to FIGS. 11, 15 and 16, FIG. 17 shows a section of a surgical instrument of a robot according to one design of the present invention. Corresponding elements are identified with the same reference signs, so that only the differences to the other depicted designs are discussed in the following, while we otherwise refer to their descriptions.

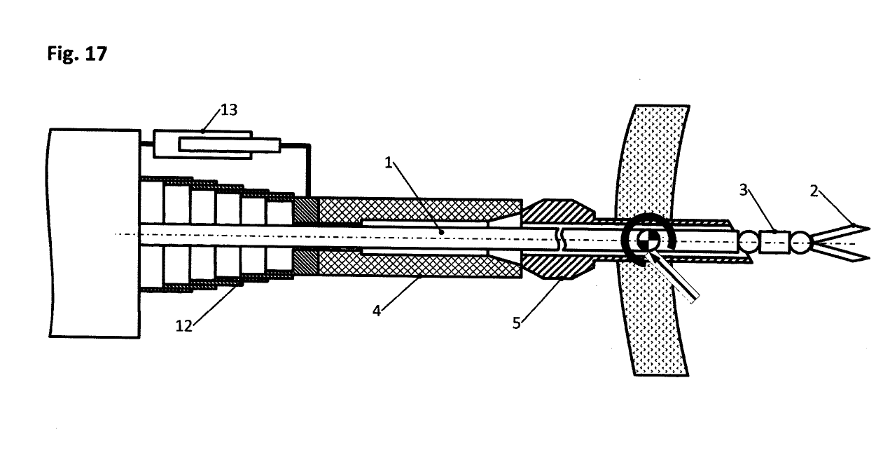

In the design of FIG. 17, the protective cap 4 is connected to a proximal region, in particular the end, of the shaft by means of a telescopic sleeve 12 surrounding the shaft 1. The telescopic sleeve 12 advantageously allows the extracorporeal section of the shaft 1 to be distally mechanically supported by the protective cap 4. Additionally or alternatively, the telescopic sleeve can protect the shaft.

In the design of FIG. 17, the telescopic sleeve comprises an optional gravity compensation mechanism 13 to offset the weight force of the trocar sleeve 5 connected to the protective cap 4. Additionally or alternatively, the protective cap 4 can be pulled back with the aid of the mechanism 13, and/or fixed in any position along the shaft axis.

### LIST OF REFERENCE SIGNS

1 Shaft
1.1 Radial collar
1.2 Longitudinal groove
1.3 Leg
2 End effector
3 Joint assembly
4 Protective cap
4.1 Guide sleeve
4.2 Cover sleeve
4a Proximal section
4b Distal section
4c Chamfer (convergent insertion opening)
4d Elliptical curvature (convergent insertion opening)
4e, 5f External cone
4f, 5e Internal cone
4g Circular bore
4h, 4h′ Radial projection
5 Trocar sleeve
6 Distal cover
6.1 (one-piece) flap
6.2, 6.4 Segment
6.3 Flap
7 Safety cable
8 Magnet
9 Locking mechanism
10 Spring
12 Telescopic sleeve
13 Mechanism

What is claimed is:

1-14. (canceled)

15. A robot with a tool, comprising:
a shaft with a distal joint assembly with at least one degree of freedom; and
a protective cap that can be moved from a distal position to a proximal position on the shaft;
wherein the protective cap accommodates the joint assembly in the distal position; and
wherein the joint assembly protrudes distally from the protective cap when the protective cap is in the proximal position.

16. The robot of claim 15, wherein the tool is a surgical instrument.

17. The robot of claim 15, further comprising a sleeve connectable with the protective cap.

18. The robot of claim 17, wherein the sleeve is a trocar sleeve.

19. The robot of claim 17, wherein the sleeve is releasably connectable with the protective cap in at least one of:
a form fitting manner;
a force-locking manner;
a frictionally engaged manner; or
a magnetic manner.

20. The robot of claim 17, wherein the protective cap and the sleeve further each include complementary guide structure that cooperate to connect the protective cap with the sleeve.

21. The robot of claim 20, wherein the complementary guide structure centers the protective cap and the sleeve.

22. The robot if claim 15, wherein the protective cap comprises a distal cover.

23. The robot of claim 22, wherein the distal cover is at least one of:
a) a single, unitary component or a multi-part component;
b) rotatable; or
c) deformable, in at least one of plastically or in sections.

24. The robot of claim 15, wherein the protective cap is configured for locking in at least one of the distal position, the proximal position, or at least one intermediate position between the distal position and the proximal position.

25. The robot of claim 24, wherein the protective cap is configured for releasably locking in at least one of:
a form fitting manner;
a force-locking manner;
a frictionally engaged manner; or
a magnetic manner.

26. The robot of claim 15, wherein the protective cap is prestressed in distal direction and/or is movably guided on the shaft in a rotatable or torque-proof manner.

27. The robot of claim 26, wherein the protective cap is movably guided on the shaft by a telescopic sleeve encircling the shaft.

28. The robot of claim 15, wherein the protective cap comprises a convergent insertion opening.

29. The robot of claim 28, wherein the convergent insertion opening is on a distal end of the protective cap.

30. The robot of claim 15, wherein the protective cap is captively bound to at least one of the shaft or the robot.

31. The robot of claim 30, wherein the protective cap is captively bound to the shaft or the robot by a telescopic sleeve encircling the shaft.

32. The robot of claim 15, wherein the protective cap comprises a guide sleeve and a cover sleeve connected to the guide sleeve.

33. The robot of claim 32, wherein the cover sleeve is releasably connected to the guide sleeve in at least one of:
a form fitting manner;
a force-locking manner;
a frictionally engaged manner; or
a magnetic manner.

34. The robot of claim 15, wherein at least part of the protective cap can be removed from the shaft.

35. The robot of claim 34, wherein the part of the protective cap this can be removed is at least one of a guide sleeve or a cover sleeve connected to the guide sleeve.

36. The robot of claim 35, wherein the cover sleeve is releasably connected to the guide sleeve in at least one of:
a form fitting manner;
a force-locking manner;
a frictionally engaged manner; or
a magnetic manner.

37. A protective cap for use with a tool of a robot, the tool including a shaft with a distal joint assembly having at least one degree of freedom:
wherein the protective cap is movable along the shaft from a distal position to a proximal position;
wherein the protective cap accommodates the joint assembly in the distal position; and
wherein the joint assembly protrudes distally from the protective cap when the protective cap is in the proximal position.

38. A method for handling a tool of a robot, the method comprising:
moving a protective cap from a distal position to a proximal position on a shaft of the tool, in front of a tool insert.

39. The method of claim 38, wherein moving the protective cap comprises moving the cap with the robot.

40. The method of claim 39, wherein moving the protective cap with the robot comprises moving the protective cap using a connection to a sleeve.

41. The method of claim 40, wherein the sleeve is releasably connected to the protective cap in at least one of:
a form fitting manner;
a force-locking manner;
a frictionally engaged manner; or
a magnetic manner.

42. The method of claim 40 wherein the sleeve is a trocar sleeve.

43. The method of claim 38, wherein the protective cap is moved from the proximal position to the distal position on the shaft of the tool, behind a tool insert.
